(19) United States
(12) Patent Application Publication
(10) Pub. No.: US 2026/0297400 A1
(43) Pub. Date: Oct. 1, 2026

(54) IMPLANTABLE MEDICAL DEVICE WITH CURABLE LIQUID FEEDTHROUGH POTTING ADHESIVE

(71) Applicant: Medtronic, Inc., Minneapolis, MN (US)

(72) Inventor: Yangbin Chen, Lake Elmo, MN (US)

(21) Appl. No.: 19/478,054

(22) PCT Filed: Mar. 26, 2024
(86) PCT No.: PCT/IB2024/052888

§ 371 Date: Oct. 23, 2025

Related U.S. Application Data
(60) Provisional application No. 63/461,410, filed on Apr. 24, 2023.

(51) Int. Cl. C09J 163/00; A61N 1/375; C09J 11/06

(52) U.S. Cl. CPC C09J 163/00; A61N 1/3754; C09J 11/06

(57) ABSTRACT

An implantable medical device with a curable liquid feedthrough potting adhesive, and methods of making the same are provided. The curable liquid feedthrough potting adhesive is capable of curing within 3 hours at an ambient temperature of 70° C. or less and may have a pot life of at least 60 hours. The curable liquid feedthrough potting adhesive bonds a feedthrough pin within a feedthrough ferrule of the implantable medical device. The curable liquid feedthrough potting adhesive includes an epoxy resin, a thiol hardener, and a solid amine catalyst.

This application claims the benefit of U.S. Provisional Patent Application Ser. No. 63/461,410, filed Apr. 24, 2023, the entire content of which is incorporated herein by reference.

### TECHNICAL FIELD

This disclosure generally relates to, among other things, potting adhesives for use in implantable medical devices and, more specifically, for curable liquid potting adhesives for use in feedthrough ferrules of implantable medical devices.

### BACKGROUND

In general, there is a need for potting adhesive compositions that can provide fast cure at relatively low temperatures, while also maintaining consistent, low viscosity at room temperature (that is, an ambient temperature of between 20° C. and 25° C. or an ambient temperature of approximately 23° C.). Such potting adhesive compositions may be useful, for example, for use in implantable medical devices such as implantable pulse generators, implantable cardioverter defibrillators, and neurostimulators.

Implantable medical devices generally use potting adhesive compositions to bond feedthrough pins within a feedthrough ferrule and to electrically insulate the pin from the ferrule. Potting adhesives may also commonly be used to isolate the internal components of the implantable medical device from the patient's body and the patient's bodily fluid. Because internal components, such as batteries, capacitors, and processors, for example, may commonly be sensitive to the aqueous environment of the patient's body, it is important to isolate sensitive internal components.

Important characteristics for feedthrough potting adhesive compositions may include, for example, viscosity, pot life, mixing compatibility of the parts (such as the mixing compatibility of a resin and a hardener), cure time, and cure temperature. To isolate the internal components of an implantable medical device, biocompatibility and durability of the feedthrough potting adhesive may also be important considerations, particularly for implantable medical devices intended for long-term use.

### SUMMARY

As described herein, curable liquid feedthrough potting adhesive compositions for implantable medical devices may be prepared with useful characteristics including low viscosity, long pot life, and room temperature mixing compatibility of the resin and the hardener, as a few examples. As another example, the adhesive compositions described herein may provide fast cure at relatively low temperatures. Fast curing at low temperatures may be useful, for example, for use in implantable medical devices. Fast curing at low temperatures may be specifically useful, for example, when curing of the adhesive occurs after temperature-sensitive internal components, such as batteries, are already in the implantable medical device. As still another example, feedthrough potting adhesive compositions described herein may provide biocompatibility for use in applications such as implantable medical devices.

Embodiments disclosed herein may include an implantable medical device feedthrough assembly with a feedthrough ferrule and a reaction product of a curable liquid feedthrough potting adhesive in the feedthrough ferrule. The adhesive includes an epoxy resin with one or more of a glycidyl ether group, an alkoxysilane group, and a nano silica. The adhesive further includes a thiol hardener with at least two thiol groups, an alkoxysilane group, or at least two thiol groups and an alkoxysilane group. The adhesive still further includes a solid catalyst comprising an amine group.

The reaction product may have a glass transition temperature of less than 50° C., less than 40° C., or between 27° C. and 47° C. The reaction product may be substantially free of water. The reaction product of the curable liquid feedthrough potting adhesive may have a water uptake of less than 5 wt-%, less than 4 wt-%, less than 3 wt-%, or less than 2 wt-% after 63 days in a 37° C. aqueous environment. The reaction product may be curable within 2 hours at an ambient temperature of less than 70° C., within 3 hours at an ambient temperature of less than 60° C., or within 4 hours at an ambient temperature of less than 50° C. The epoxy resin may be non-polar and may have an octanol-water partition coefficient (log P) of great than 2. The thiol hardener may be non-polar and may have an octanol-water partition coefficient (log P) of great than 2. The curable liquid feedthrough potting adhesive may include less than 5% of the solid catalyst, less than 3% of the solid catalyst, or less than 2% of the solid catalyst.

Embodiments described herein may further include a method for forming an implantable medical device, the method including bonding a feedthrough pin within a ferrule with a curable liquid feedthrough potting adhesive and curing the curable liquid feedthrough potting adhesive. The adhesive may include an epoxy resin with one or more of a glycidyl ether group, an alkoxysilane group, and a nano silica; a thiol hardener with at least two thiol groups, an alkoxysilane group, or at least two thiol groups and an alkoxysilane group; and a solid catalyst with an amine group.

Curing the curable liquid potting adhesive may occur within 2 hours at an ambient temperature of less than 70° C., within 3 hours at an ambient temperature of less than 60° C., or within 4 hours at an ambient temperature of less than 50° C. The curing may form a cured potting adhesive with a glass transition temperature of less than 50° C., less than 40° C., or between 27° C. and 47° C. The curing may form a cured potting adhesive substantially free of water. The curing may form a cured potting adhesive with a water uptake of less than 5 wt-%, less than 4 wt-%, less than 3 wt-%, or less than 2 wt-% after 63 days in a 37° C. aqueous environment. The epoxy resin may be non-polar and may have an octanol-water partition coefficient (log P) of greater than 2. The thiol hardener may be non-polar and may have an octanol-water partition coefficient (log P) of greater than 2. The curable liquid feedthrough potting adhesive may include less than 5% of the solid catalyst, less than 3% of the solid catalyst, or less than 2% of the solid catalyst.

Embodiments described herein may still further include a curable liquid feedthrough potting adhesive suitable for use in a feedthrough ferrule of an implantable medical device, capable of curing within 3 hours at an ambient temperature of 70° C. or less, and with a pot life of at least 6 hours. The curable liquid feedthrough potting adhesive may include an epoxy resin with one or more of a glycidyl ether group, an alkoxysilane group, and a nano silica; a thiol hardener with two or more thiol groups, an alkoxysilane group, or two or more thiol groups and an alkoxysilane group; and a solid catalyst with an amine group.

The adhesive may be substantially free of water. A reaction product of the epoxy resin, the thiol hardener, and the solid catalyst, when cured, may have a water uptake of less than 5 wt-%, less than 4 wt-%, less than 3 wt-%, or less than 2 wt-% after 63 days in a 37° C. aqueous environment. The epoxy resin may be non-polar and may have an octanol-water partition coefficient (log P) of greater than 2. The thiol hardener may be non-polar and may have an octanol-water partition coefficient (log P) of greater than 2. The adhesive may include less than 15% polar compounds and may have a pH of greater than 7. The adhesive may include less than 5%, less than 3%, or less than 2% of the solid catalyst.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is an illustrative implantable medical device implanted in a patient including a curable liquid feedthrough potting adhesive.

FIG. 2 is a cross-section side view of an illustrative feedthrough assembly including the curable liquid feedthrough potting adhesive of the implantable medical device of FIG. 1.

FIG. 3 is a schematic of an illustrative method for preparing the curable liquid feedthrough potting adhesive of FIG. 2.

FIG. 4 is a graphical representation showing the pot time of the curable liquid feedthrough potting adhesive of FIG. 2.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various exemplary embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art unless, otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “substantially” modifies the term that follows by at least about 90%, at least about 95%, or at least about 98%. “Substantially” includes “significantly,” which refers to statistical significance.

The term “not substantially” modifies the term that follows by not more than 25%, not more than 10%, not more than 5%, or not more than 2%.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to”, “at most”, or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Such inclusive or open-ended words encompass more restrictive or closed terms or phrases, such as “consisting” or “consisting essentially.”

As used herein, “consisting essentially of” means that the article or method consisting essentially of listed elements may include additional elements that do not materially affect the basic and novel characteristics of the article or method.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Reference in the specification to “some embodiments,” “an embodiment,” “one embodiment,” “embodiments,” “one or more embodiments,” or “other embodiments” means that a particular feature, structure, or characteristic described in connection with the embodiments is included in at least some embodiments, but not necessarily all embodiments, of the present disclosures.

Any direction referred to herein, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

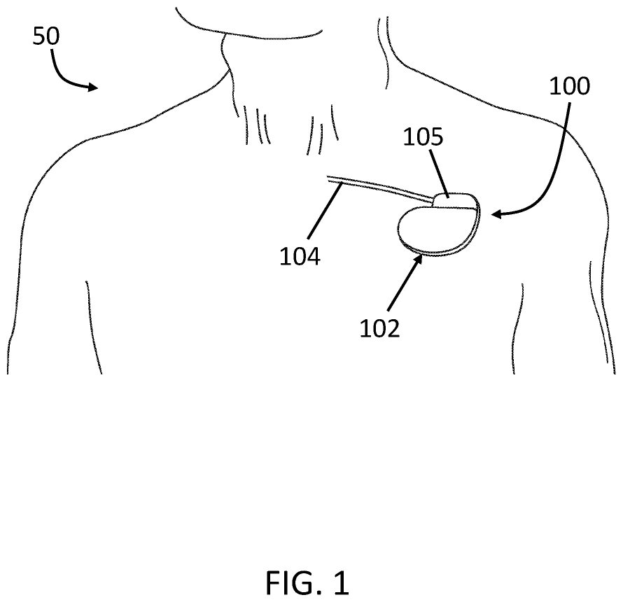

As described herein, a curable liquid feedthrough potting adhesive composition may be prepared, for example, for use in a feedthrough ferrule of an implantable medical device. An illustrative implantable medical device 100 implanted in a patient 50 including the curable liquid feedthrough potting adhesive is shown in FIG. 1. The implantable medical device 100 may include a case 102, which may also be referred to as a housing. The case 102 may house one or more internal components of the implantable medical device 100, such as one or more batteries, electrical circuits, and the like. The implantable medical device 100 may further include one or more external components, such as a lead 104. The one or more external components may be electrically connectable to the one or more internal components via a feedthrough, which may be positioned in a header 105. The header 105 may form a unitary part with, or may be coupled to, the case 102.

The header 105 may define a bore through which a portion of the lead 104 may be inserted. The lead 104 may have electrical contacts that electrically couple with contacts in the header 105 when the lead 104 is inserted into the bore. The electrical contacts in the header 105 may be electrically coupled to the electrical components of the implantable medical device 100 through one or more feedthroughs.

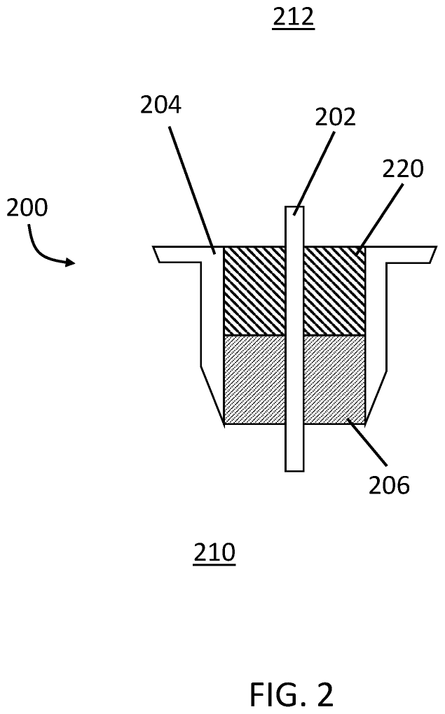

An illustrative feedthrough assembly 200 including the curable liquid feedthrough potting adhesive 220 is shown in FIG. 2. The feedthrough assembly 200 may include a feedthrough pin 202 extending and providing electrical connection between an internal volume 210 (such as within the case 102 shown in FIG. 1) of an implantable medical device and an outside environment 212 (such as within the header 105 shown in FIG. 1, which may define a bore in communication with a patient's bodily fluid). The feedthrough assembly 200 may include a ferrule 204 having at least a portion disposed between the internal volume 210 and the outside environment 212. The ferrule 204 may be tubular. The feedthrough pin 202 may extend through the ferrule 204.

The feedthrough assembly 200 may include a plug element 206 disposed at least partially within the ferrule 204. The feedthrough pin 202 may extend through a lumen in the plug element 206.

The feedthrough assembly 200 may include the curable liquid feedthrough potting adhesive 220. The adhesive 220 may be disposed in the ferrule 204. The pin 202 may extend through the adhesive 220. The adhesive 220 may provide a bond between the pin 202 and the ferrule 204. The adhesive 220, as described herein, may provide a seal between the outside environment 212 and the internal volume 210. The adhesive 220 may additionally or alternatively provide electrical insulation between the ferrule 204 and the pin 202.

Illustrative embodiments of the curable adhesive composition may include an epoxy resin and a thiol hardener. The curable adhesive composition may further include a solid catalyst. The composition of the curable adhesive may be selected for characteristics such as low viscosity, long pot life, and fast curing at low temperature, as examples. The epoxy resin and the thiol hardener may be selected for characteristics such as mixing compatibility at room temperature, as an example.

In one or more embodiments according to the present disclosure, the curable liquid potting adhesive may include an epoxy resin. The curable liquid potting adhesive may include any suitable epoxy resin. Suitable epoxy resins may be selected based on polarity, room-temperature mixing compatibility with the hardener, and viscosity, as a few examples.

In one or more embodiments, the epoxy resin may be non-polar, substantially non-polar, hydrophobic, or substantially hydrophobic, or the epoxy resin may include compounds that are non-polar, substantially non-polar, hydrophobic, or substantially hydrophobic. The epoxy resin or compounds included in the epoxy resin may have any suitable octanol-water partition coefficient (Log P). Suitable epoxy resin octanol-water partition coefficients may include, for example, between 2 and 15 or between 3 and 10. In further examples, suitable octanol-water partition coefficients may include greater than 1.5, greater than 2, greater than 3, greater than 5, or greater than 10. The octanol-water partition coefficient may be determined according to ASTM E1147-92 (1997), “Standard Test Method for Partition Coefficient (N-Octanol/Water) Estimation by Liquid Chromatography.”

In some embodiments, suitable epoxy resins may include compounds with one or more glycidyl ether groups. Suitable epoxy resin compounds with one or more glycidyl ether groups may include, for example, mono- or multi-glycidyl ether of phenols, and mono- or multi-glycidyl ether of aromatic, aliphatic, and cycloaliphatic alcohols.

The curable liquid feedthrough potting adhesive may include epoxy resin compounds with one or more glycidyl ether groups in any suitable concentration. Suitable concentrations of epoxy resin compounds with one or more glycidyl ether groups may be between 15 wt-% and 75 wt-% or between 20 wt-% and 70 wt-%, as examples. In further examples, suitable concentrations of epoxy resin compounds with one or more glycidyl ether groups may include greater than 15 wt-%, greater than 25 wt-%, greater than 35 wt-%, greater than 45 wt-%, greater than 55 wt-%, greater than 65 wt-%, or greater than 75 wt-%. Additionally or alternatively, suitable concentrations of epoxy resin compounds with one or more glycidyl ether groups may include less than 80 wt-%, less than 70 wt-%, less than 60 wt-%, less than 50 wt-%, less than 40 wt-%, less than 30 wt-%, or less than 20 wt-%.

In embodiments according to the present disclosure, suitable epoxy resins may include compounds with one or more alkoxysilane groups. Suitable epoxy resin compounds with one or more alkoxysilane groups may include, for example, (3-Glycidyloxypropyl)trimethoxysilane, (3-Glycidyloxypropyl)triethoxysilane, 5,6-Epoxyhexyltriethoxysilane, (3-Glycidoxypropyl)methyldiethoxysilane, (3-Glycidoxypropyl)methyldimethoxysilane, and (3-Glycidoxypropyl)dimethylethoxysilane.

The curable liquid feedthrough potting adhesive may include epoxy resin compounds with one or more alkoxysilane groups in any suitable concentration. Suitable concentrations of epoxy resin compounds with one or more alkoxysilane groups may be between 0.05 wt-% and 10 wt-% or between 0.1 wt-% and 5 wt-%, as examples. In further examples, suitable concentrations of epoxy resin compounds with one or more alkoxysilane groups may include greater than 0.05 wt-%, greater than 0.1 wt-%, greater than 0.5 wt-%, greater than 1 wt-%, greater than 2.5 wt-%, greater than 4 wt-%, or greater than 5 wt-%. Additionally or alternatively, suitable concentrations of epoxy resin compounds with one or more alkoxysilane groups may include less than 10 wt-%, less than 7 wt-%, less than 5 wt-%, less than 3 wt-%, less than 1 wt-%, less than 0.6 wt-%, less than 0.3 wt-%, or less than 0.1 wt-%.

In additional or alternative embodiments according to the present disclosure, suitable epoxy resins may include a nano silica, such as nano silica filled epoxies, for example. Suitable epoxy resins with a nano silica may include, for example, NANOPOX F 400 (available from EVONIK), NANOPOX F 440 (available from EVONIK), NANOPOX F 520 (available from EVONIK), and NANOPOX F 700 (available from EVONIK).

The curable liquid feedthrough potting adhesive may include epoxy resins with a nano silica in any suitable concentration. Suitable concentrations of epoxy resins with a nano silica may be between 1 wt-% and 75 wt-% or between 5 wt-% and 60 wt-%, as examples. In further examples, suitable concentrations of epoxy resins with a nano silica may include greater than 1 wt-%, greater than 5 wt-%, greater than 15 wt-%, greater than 35 wt-%, greater than 50 wt-%, or greater than 60 wt-%. Additionally or alternatively, suitable concentrations of epoxy resins with a nano silica may include less than 75 wt-%, less than 60 wt-%, less than 45 wt-%, less than 35 wt-%, less than 15 wt-%, or less than 5 wt-%.

In one or more embodiments according to the present disclosure, the curable liquid feedthrough potting adhesive may include a thiol hardener. The curable liquid feedthrough potting adhesive may include any suitable thiol hardener. Suitable thiol hardeners may be selected based on polarity, room-temperature mixing compatibility with the epoxy resin, and viscosity, as a few examples.

In one or more embodiments, the thiol hardener may be non-polar, substantially non-polar, hydrophobic, or substantially hydrophobic, or the thiol hardener may include compounds that are non-polar, substantially non-polar, hydrophobic, or substantially hydrophobic. The thiol hardener, or compounds included in the thiol hardener, may have any suitable octanol-water partition coefficient (log P). Suitable thiol hardener octanol-water partition coefficients may include, for example, between 2 and 15 or between 3 and 10. In further examples, suitable thiol hardener octanol-water partition coefficients may include greater than 1.5, greater than 2, greater than 3, greater than 5, or greater than 10.

In some embodiments, suitable thiol hardeners may include compounds with two or more thiol groups. Suitable thiol hardener compounds with two or more thiol groups may include, for example, Trimethylolpropane tris(3-mercaptopropionate), Pentaerythritol tetrakis(3-mercaptopropionate), Poly(ethylene glycol)dithiol, Dipentaerythritol Hexakis (3-mercaptopropionate), 1,3,5-tris(mercaptomethyl)benzene, Tris [2-(3-mercaptopropionyloxy)ethyl] isocyanurate, Ethylene Glycol Bis(3-mercaptopropionate), and Ethoxylated trimethylolpropane tri (3-mercaptopropionate).

The curable liquid feedthrough potting adhesive may include thiol hardener compounds with two or more thiol groups in any suitable concentration. Suitable concentrations of thiol hardener compounds with two or more thiol groups may be between 20 wt-% and 80 wt-% or between 30 wt-% and 60 wt-%, as examples. In further examples, suitable concentrations of thiol hardener compounds with two or more thiol groups may include greater than 20 wt-%, greater than 30 wt-%, greater than 40 wt-%, greater than 50 wt-%, greater than 60 wt-%, or greater than 70 wt-%. Additionally or alternatively, suitable concentrations of thiol hardener compounds with two or more thiol groups may include less than 80 wt-%, less than 70 wt-%, less than 60 wt-%, less than 50 wt-%, less than 40 wt-%, or less than 30 wt-%.

In some embodiments, suitable thiol hardeners may include compounds with one or more alkoxysilane groups. Suitable thiol hardener compounds with one or more alkoxysilane groups may include, for example, 3-Mercaptopropyltrimethoxysilane, 3-Mercaptopropyltriethoxysilane, 11-Mercaptoundecyltrimethoxysilane, and 3-Mercaptopropylmethyldimethoxysilane.

The curable liquid feedthrough potting adhesive may include thiol hardener compounds with one or more alkoxysilane groups in any suitable concentration. Suitable concentrations of thiol hardener compounds with one or more alkoxysilane groups may be between 0.05 wt-% and 10 wt-% or between 0.1 wt-% and 5 wt-%, as examples. In further examples, suitable concentrations of thiol hardener compounds with one or more alkoxysilane groups may include greater than 0.05 wt-%, greater than 0.1 wt-%, greater than 0.5 wt-%, greater than 1 wt-%, greater than 2.5 wt-%, greater than 4 wt-%, or greater than 5 wt-%. Additionally or alternatively, suitable concentrations of thiol hardener compounds with one or more alkoxysilane groups may include less than 7 wt-%, less than 5 wt-%, less than 3 wt-%, less than 1 wt-%, less than 0.6 wt-%, less than 0.3 wt-%, or less than 0.1 wt-%.

In one or more embodiments according to the present disclosure, the curable liquid feedthrough potting adhesive may include a solid catalyst. The curable feedthrough liquid potting adhesive may include any suitable solid catalyst. Suitable solid catalysts may be selected based on room-temperature solubility in the liquid adhesive mixture, as an example. Solid catalysts with low room-temperature solubility in the liquid adhesive mixture may be beneficial because un-dissolved solid catalyst may be unavailable to catalyze reaction between the epoxy resin and the thiol hardener. In other words, dissolution of the solid catalyst in the liquid adhesive mixture may make the solid catalyst available to catalyze the curing reaction, which may cause viscosity of the liquid adhesive mixture to increase. That is to say, solid catalysts with low room-temperature solubility in the liquid adhesive mixture may beneficially increase pot life of the curable liquid feedthrough potting adhesive. As another example, suitable solid catalysts may be selected based on particle size. Solid catalysts with small particle size (for example, less than 50 microns, less than 25 microns, or less than 10 microns) may be beneficial, for example, because smaller particles are more readily dispersed in the liquid feedthrough potting adhesive mixture.

In some embodiments, suitable solid catalysts may include compounds with amine functionality, such as compounds including one or more amine groups. Examples of suitable amine groups may include, for example, one or more primary amines, one or more secondary amines, one or more tertiary amines, or combinations thereof.

Suitable solid catalyst compounds with amine functionality may include, for example, AJICURE PN-23J (available from A&C CATALYSTS INC.), AJICURE PN-31J (available from A&C CATALYSTS INC.), AJICURE MY-24 (available from A&C CATALYSTS INC.), AJICURE VDH-J (available from A&C CATALYSTS INC.), ANCAMINE 2014AS (available from AIR PRODUCTS), ANCAMINE 2014FG (available from AIR PRODUCTS), ANCAMINE 2337S (available from AIR PRODUCTS), ANCAMINE 2441 (available from AIR PRODUCTS), ANCAMINE 2442 (available from AIR PRODUCTS), FUJICURE FXR-1020 (available from T&K TOKA), FUJICURE FXR-1030 (available from T&K TOKA), FUJICURE FXR-1081 (available from T&K TOKA), FUJICURE FXR-1121 (available from T&K TOKA), EH-5030S (available from ADEKA), EH-5057P (available from ADEKA), CUREZOL SIZ (available from SHIKOKU), CUREZOL 2MZ-H (available from SHIKOKU), CUREZOL C11Z (available from SHIKOKU), CUREZOL C17Z (available from SHIKOKU), CUREZOL 2PZ (available from SHIKOKU), and CUREZOL 2P4MZ (available from SHIKOKU).

The curable liquid feedthrough potting adhesive may include the solid catalyst in any suitable concentration. Suitable concentrations of the solid catalyst may be between 0.5 wt-% and 20 wt-% or between 1 wt-% and 15 wt-%, as examples. In further examples, suitable concentrations of the solid catalyst may include greater than 0.5 wt-%, greater than 1 wt-%, greater than 5 wt-%, greater than 10 wt-%, greater than 15 wt-%, or greater than 20 wt-%. Additionally or alternatively, suitable concentrations of the solid catalyst may include less than 20 wt-%, less than 10 wt-%, less than 5 wt-%, less than 3 wt-%, less than 2 wt-%, less than 1.5 wt-%, or less than 1 wt-%.

The curable liquid feedthrough potting adhesive may have any suitable viscosity at room temperature. Suitable viscosities at room temperature may be selected based on the flow and wetting characteristics desirable for effective flow and wetting of a feedthrough ferrule, as an example. As another example, suitable viscosities at room temperature may be selected based on capability of the curable liquid feedthrough potting adhesive to be dispensed through a needle. Suitable viscosities at room temperature may be between 500 centipoise (cps) and 3,000 cps or between 1,000 cps and 2,500 cps, as examples. Further examples of suitable viscosities at room temperature may include greater than 500 cps, greater than 800 cps, greater than 1,000 cps, greater than 1,500 cps, greater than 1,700 cps, greater than 2,000 cps, or greater than 2,500 cps. Additionally or alternatively, suitable viscosities at room temperature may include, for examples, less than 3,000 cps, less than 2,500 cps, less than 2,000 cps, less than 1,800 cps, less than 1,500 cps, or less than 1,000 cps. The curable liquid feedthrough potting adhesive may have a viscosity at room temperature of approximately 1,500 cps.

The curable liquid feedthrough potting adhesive may have any suitable pH value. Suitable pH values may be selected based on low solubility of the solid catalyst at ambient temperature, as an example. Suitable pH values may include, for example, about 7.5. Other examples of suitable pH values may include between 7 and 8.

In one or more embodiments, the curable liquid feedthrough potting adhesive may be non-polar, substantially non-polar, hydrophobic, or substantially hydrophobic. As described herein, non-polarity or hydrophobicity of the curable liquid feedthrough potting adhesive and the reaction product of the curable liquid feedthrough potting adhesive (that is, the cured feedthrough potting adhesive) may be beneficial, for example, to reduce water uptake (such as water uptake of the cured potting adhesive) or to reduce dissolution of the solid catalyst at room temperature.

In one or more embodiments, the curable liquid feedthrough potting adhesive may have a long pot life. The pot life of the curable liquid feedthrough potting adhesive may be described as the time period after mixing together the epoxy resin, the thiol hardener, and the solid catalyst in which the adhesive mixture is useable. Pot life may commonly be limited by the viscosity of an adhesive mixture increasing over time spent at room temperature. As described herein, viscosity may increase, for example, as a result of the solid catalyst dissolving in the adhesive mixture. The pot life of the curable liquid feedthrough potting adhesive may be defined by the length of time after mixing in which the adhesive has a viscosity of less than 2,500 cps or less than 2,000 cps, as just two examples. The curable liquid feedthrough potting adhesive may have a viscosity of less than 2,000 cps at room temperature for more than 8 hours or for more than 24 hours, as examples. For further examples, the curable liquid feedthrough potting adhesive may have a viscosity of less than 2,500 cps at room temperature for more than 8 hours or for more than 24 hours.

The curable liquid feedthrough potting adhesive may have any suitable pot life. For example, the curable liquid feedthrough potting adhesive may have a viscosity of less than 2,000 cps at room temperature for more than 6 hours, more than 10 hours, more than 24 hours, more than 48 hours, more than 70 hours, more than 100 hours, more than 120 hours, more than 150 hours, more than 180 hours, more than 200 hours, more than 250 hours, more than 300 hours, or more than 500 hours. The curable liquid feedthrough potting adhesive may additionally or alternatively have a viscosity of less than 2,500 cps at room temperature for more than 6 hours, more than 10 hours, more than 24 hours, more than 48 hours, more than 70 hours, more than 100 hours, more than 120 hours, more than 150 hours, more than 180 hours, more than 200 hours, more than 250 hours, more than 300 hours, or more than 500 hours.

In some embodiments, the curable liquid feedthrough potting adhesive may be capable of curing relatively quickly at a relatively low ambient temperature. For example, the adhesive may be capable of curing within 3 hours at less than 70° C. Fast curing at a relatively low ambient temperature may have advantages including reducing energy consumption used to cure the adhesive and improved manufacturing efficiency, as examples. As another example, advantages of fast curing at a relatively low ambient temperature may include improving the quality of internal components of a finished implantable medical device, such as the quality of batteries therein, which may be subjected to the curing conditions. In some embodiments, the adhesive may be capable of curing within 1 hour at approximately 50° C. In additional or alternative embodiments, the adhesive may be capable of curing within 2 hours at approximately 60° C.

The adhesive may be capable of curing at any suitable temperature. Suitable curing temperatures may include, for example, less than 40° C., less than 50° C., less than 60° C., less than 70° C., less than 80° C., or less than 90° C. Suitable curing temperatures may additionally or alternatively include, for example, greater than 30° C., greater than 35° C., greater than 40° C., greater than 45° C., greater than 55° C., greater than 65° C., or greater than 75° C.

The curable liquid feedthrough potting adhesive may be capable of curing within any suitable time period. Suitable cure times may include, for example, less than 4 hours, less than 3.5 hours, less than 3 hours, less than 2.5 hours, less than 2 hours, less than 1.5 hours, less than 1 hour, or less than 30 minutes. Suitable curing times may additionally or alternatively include, for example, greater than 20 minutes, greater than 45 minutes, greater than 1 hour, greater than 2 hours, greater than 2.5 hours, greater than 3 hours, or greater than 4 hours.

One or more embodiments of the present disclosure may include the reaction product of the curable liquid potting adhesive. That is, a cured potting adhesive. The cured potting adhesive may have low water uptake. That is, the cured potting adhesive may be resistant to adsorption, or absorption, of water from the environment. Put another way, the cured potting adhesive may be non-hygroscopic.

Low water uptake may be beneficial, for example, once an implantable medical device including the cured potting adhesive is implanted into human body, and the cured potting adhesive is exposed to an aqueous environment at an ambient temperature of approximately 37° C. In an implantable medical device including the cured potting adhesive, it may be beneficial for the cured potting adhesive to have low water uptake, for example, to maintain electrical insulation between the ferrule and the pin of the feedthrough assembly.

The cured potting adhesive may have any suitable water uptake. Suitable water uptake may be based on, for example, maintaining electrical insulation between a pin (such as the pin 202) and a ferrule (such as the ferrule 204) of an implantable medical device (such as the implantable medical device 100). Suitable water uptake may be between 1 wt-% and 3 wt-%, as an example. Suitable water uptakes may include, for another example, approximately 2 wt-%. Suitable water uptakes may additionally or alternatively include, for further examples, less than 3 wt-%, less than 2.5 wt-%, less than 2.3 wt-%, less than 2 wt-%, less than 1.5 wt-%, or less than 1 wt-%. Water uptake may be determined after soaking in 37° C. water for 63 days.

Low water uptake may be affected by or based on characteristics such as hydrophobicity and non-polarity of compounds in the curable liquid feedthrough potting adhesive and compounds in the cured feedthrough potting adhesive, as just two examples. For example, the cured potting adhesive may be substantially non-polar. That is to say, the compounds included in the cured potting adhesive may be non-polar or substantially non-polar. In such embodiments, the components of the cured potting adhesive may be non-polar or substantially non-polar.

The cured potting adhesive may have any suitable glass transition temperature. Suitable glass transition temperatures may be selected based on normal body temperature, as an example. Suitable glass transition temperatures may include, for example, approximately 37° C., between 20° C. and 50° C., or between 27° C. and 47° C. Other examples of suitable glass transition temperatures may include less than 55° C., less than 45° C., less than 35° C. or less than 25° C., and/or greater than 20° C., greater than 30° C., greater than 40° C., or greater than 50° C.

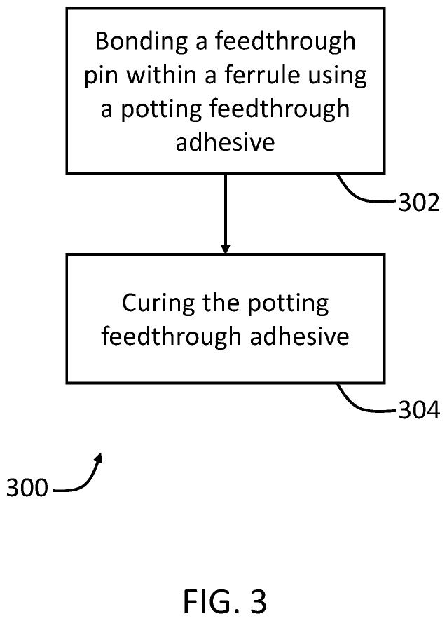

An illustrative method 300 for forming an implantable medical device using the curable liquid feedthrough potting adhesive is shown in FIG. 3. The method 300 may include bonding 302 a feedthrough pin of the implantable medical device within a ferrule of the implantable medical device. The bonding 302 may be done with a curable liquid feedthrough potting adhesive according to one or more embodiments described herein.

The method 300 may include curing 304 the curable liquid feedthrough potting adhesive. The curing 304 may occur at any suitable temperature, as described herein.

### ILLUSTRATIVE ASPECTS

The following is a list of illustrative embodiments according to the present disclosure.
Aspect 1 is an implantable medical device feedthrough assembly comprising: a feedthrough ferrule; and a reaction product of a curable liquid feedthrough potting adhesive in the feedthrough ferrule, the adhesive comprising:
   an epoxy resin comprising one or more of a glycidyl ether group, an alkoxysilane group, and a nano silica;
   a thiol hardener comprising at least two thiol groups, an alkoxysilane group, or at least two thiol groups and an alkoxysilane group; and
   a solid catalyst comprising an amine group.
Aspect 2 is the implantable medical device feedthrough assembly of aspect 1, wherein the reaction product has a glass transition temperature of less than 50° C.
Aspect 3 is the implantable medical device feedthrough assembly of aspect 1, wherein the reaction product has a glass transition temperature of less than 40° C.
Aspect 4 is the implantable medical device feedthrough assembly of aspect 1, wherein the reaction product has a glass transition temperature of between 27° C. and 47° C.
Aspect 5 is the implantable medical device feedthrough assembly of aspect 1, wherein the reaction product is substantially free of water.
Aspect 6 is the implantable medical device feedthrough assembly of aspect 1, wherein the reaction product of the curable liquid feedthrough potting adhesive has a water uptake of less than 5 wt-% after 63 days in a 37° C. aqueous environment.
Aspect 7 is the implantable medical device feedthrough assembly of aspect 1, wherein the reaction product of the curable liquid feedthrough potting adhesive has a water uptake of less than 4 wt-% after 63 days in a 37° C. aqueous environment.
Aspect 8 is the implantable medical device feedthrough assembly of aspect 1, wherein the reaction product of the curable liquid feedthrough potting adhesive has a water uptake of less than 3 wt-% after 63 days in a 37° C. aqueous environment.
Aspect 9 is the implantable medical device feedthrough assembly of aspect 1, wherein the reaction product of the curable liquid feedthrough potting adhesive has a water uptake of less than 2 wt-% after 63 days in a 37° C. aqueous environment.
Aspect 10 is the implantable medical device feedthrough assembly of aspect 1, wherein the reaction product is curable within 2 hours at an ambient temperature of less than 70° C.
Aspect 11 is the implantable medical device feedthrough assembly of aspect 1, wherein the reaction product is curable within 3 hours at an ambient temperature of less than 60° C.
Aspect 12 is the implantable medical device feedthrough assembly of aspect 1, wherein the reaction product is curable within 4 hours at an ambient temperature of less than 50° C.
Aspect 13 is the implantable medical device feedthrough assembly of aspect 1, wherein the epoxy resin is non-polar.
Aspect 14 is the implantable medical device feedthrough assembly of aspect 1, wherein the epoxy resin has an octanol-water partition coefficient (log P) of greater than 2.
Aspect 15 is the implantable medical device feedthrough assembly of aspect 1, wherein the thiol hardener is non-polar.
Aspect 16 is the implantable medical device feedthrough assembly of aspect 1, wherein the thiol hardener has an octanol-water partition coefficient (log P) of greater than 2.
Aspect 17 is the implantable medical device feedthrough assembly of aspect 1, wherein the curable liquid feedthrough potting adhesive comprises less than 5% of the solid catalyst.
Aspect 18 is the implantable medical device feedthrough assembly of aspect 1, wherein the curable liquid feedthrough potting adhesive comprises less than 3% of the solid catalyst.
Aspect 19 is the implantable medical device feedthrough assembly of aspect 1, wherein the curable liquid feedthrough potting adhesive comprises less than 2% of the solid catalyst.
Aspect 20 is a method for forming an implantable medical device, the method comprising:
bonding a feedthrough pin within a ferrule with a curable liquid feedthrough potting adhesive, the adhesive comprising:
   (i) an epoxy resin comprising one or more of a glycidyl ether group, an alkoxysilane group, and a nano silica;
   (ii) a thiol hardener comprising at least two thiol groups, an alkoxysilane group, or at least two thiol groups and an alkoxysilane group; and
   (iii) a solid catalyst comprising an amine group; and curing the curable liquid feedthrough potting adhesive.
Aspect 21 is the method of aspect 20, wherein the curing the curable liquid potting adhesive occurs within 2 hours at an ambient temperature of less than 70° C.
Aspect 22 is the method of aspect 20, wherein the curing the curable liquid potting adhesive occurs within 3 hours at an ambient temperature of less than 60° C.
Aspect 23 is the method of aspect 20, wherein the curing the curable liquid potting adhesive occurs within 4 hours at an ambient temperature of less than 50° C.
Aspect 24 is the method of aspect 20, wherein the curing the curable liquid potting adhesive forms a cured potting adhesive with a glass transition temperature of less than 50° C.
Aspect 25 is the method of aspect 20, wherein the curing the curable liquid potting adhesive forms a cured potting adhesive with a glass transition temperature of less than 40° C.
Aspect 26 is the method of aspect 20, wherein the curing the curable liquid potting adhesive forms a cured potting adhesive with a glass transition temperature of between 27° C. and 47° C.
Aspect 27 is the method of aspect 20, wherein the curing the curable liquid potting adhesive forms a cured potting adhesive substantially free of water.
Aspect 28 is the method of aspect 20, wherein the curing the curable liquid potting adhesive forms a cured potting adhesive with a water uptake of less than 5 wt-% after 63 days in a 37° C. aqueous environment.
Aspect 29 is the method of aspect 20, wherein the curing the curable liquid potting adhesive forms a cured potting adhesive with a water uptake of less than 4 wt-% after 63 days in a 37° C. aqueous environment.
Aspect 30 is the method of aspect 20, wherein the curing the curable liquid potting adhesive forms a cured potting adhesive with a water uptake of less than 3 wt-% after 63 days in a 37° C. aqueous environment.
Aspect 31 is the method of aspect 20, wherein the curing the curable liquid potting adhesive forms a cured potting adhesive with a water uptake of less than 2 wt-% after 63 days in a 37° C. aqueous environment.
Aspect 32 is the method of aspect 20, wherein the curing the curable liquid potting adhesive forms a cured potting adhesive curable within 2 hours at an ambient temperature of less than 70° C.
Aspect 33 is the method of aspect 20, wherein the curing the curable liquid potting adhesive forms a cured potting adhesive curable within 3 hours at an ambient temperature of less than 60° C.
Aspect 34 is the method of aspect 20, wherein the curing the curable liquid potting adhesive forms a cured potting adhesive curable within 4 hours at an ambient temperature of less than 50° C.
Aspect 35 is the method of aspect 20, wherein the epoxy resin is non-polar.
Aspect 36 is the method of aspect 20, wherein the epoxy resin has an octanol-water partition coefficient (log P) of greater than 2.
Aspect 37 is the method of aspect 20, wherein the thiol hardener is non-polar.
Aspect 38 is the method of aspect 20, wherein the thiol hardener has an octanol-water partition coefficient (log P) of greater than 2.
Aspect 39 is the method of aspect 20, wherein the curable liquid feedthrough potting adhesive comprises less than 5% of the solid catalyst.
Aspect 40 is the method of aspect 20, wherein the curable liquid feedthrough potting adhesive comprises less than 3% of the solid catalyst.
Aspect 41 is the method of aspect 20, wherein the curable liquid feedthrough potting adhesive comprises less than 2% of the solid catalyst.
Aspect 42 is a curable liquid feedthrough potting adhesive suitable for use in a feedthrough ferrule of an implantable medical device, capable of curing within 3 hours at an ambient temperature of 70° C. or less, and with a pot life of at least 6 hours, the curable liquid feedthrough potting adhesive comprising:
an epoxy resin comprising one or more of a glycidyl ether group, an alkoxysilane group, and a nano silica;
a thiol hardener comprising two or more thiol groups, an alkoxysilane group, or two or more thiol groups and an alkoxysilane group; and
a solid catalyst comprising an amine group.
Aspect 43 is the curable liquid feedthrough potting adhesive of aspect 42, wherein the adhesive is substantially free of water.
Aspect 44 is the curable liquid feedthrough potting adhesive of aspect 42, wherein a reaction product of the epoxy resin, the thiol hardener, and the solid catalyst, when cured, has a water uptake of less than 5 wt-% after 63 days in a 37° C. aqueous environment.
Aspect 45 is the curable liquid feedthrough potting adhesive of aspect 42, wherein a reaction product of the epoxy resin, the thiol hardener, and the solid catalyst, when cured, has a water uptake of less than 4 wt-% after 63 days in a 37° C. aqueous environment.
Aspect 46 is the curable liquid feedthrough potting adhesive of aspect 42, wherein a reaction product of the epoxy resin, the thiol hardener, and the solid catalyst, when cured, has a water uptake of less than 3 wt-% after 63 days in a 37° C. aqueous environment.
Aspect 47 is the curable liquid feedthrough potting adhesive of aspect 42, wherein a reaction product of the epoxy resin, the thiol hardener, and the solid catalyst, when cured, has a water uptake of less than 2 wt-% after 63 days in a 37° C. aqueous environment.
Aspect 48 is the curable liquid feedthrough potting adhesive of aspect 42, wherein the epoxy resin is non-polar.
Aspect 49 is the curable liquid feedthrough potting adhesive of aspect 42, wherein the epoxy resin has an octanol-water partition coefficient (log P) of greater than 2.
Aspect 50 is the curable liquid feedthrough potting adhesive of aspect 42, wherein the thiol hardener is non-polar.
Aspect 51 is the curable liquid feedthrough potting adhesive of aspect 42, wherein the thiol hardener has an octanol-water partition coefficient (log P) of greater than 2.
Aspect 52 is the curable liquid feedthrough potting adhesive of aspect 42, wherein the adhesive comprises less than 15% polar compounds.
Aspect 53 is the curable liquid feedthrough potting adhesive of aspect 42, wherein the adhesive has a pH of greater than 7.
Aspect 54 is the curable liquid feedthrough potting adhesive of aspect 42, comprising less than 5% of the solid catalyst.
Aspect 55 is the curable liquid feedthrough potting adhesive of aspect 42, comprising less than 3% of the solid catalyst.
Aspect 56 is the curable liquid feedthrough potting adhesive of aspect 42, comprising less than 2% of the solid catalyst.
Aspect 57 is the curable liquid feedthrough potting adhesive of aspect 42, wherein the adhesive is capable of curing within 2 hours at an ambient temperature of less than 60° C.
Aspect 58 is the curable liquid feedthrough potting adhesive of aspect 42, wherein the adhesive has a viscosity of less than 2,000 cps at an ambient temperature of between 20° C. and 25° C.
Aspect 59 is the curable liquid feedthrough potting adhesive of aspect 42, wherein the adhesive has a viscosity of less than 2,500 cps after 8 hours at an ambient temperature of between 20° C. and 25° C.

### EXAMPLES

TABLE 1
Materials used in the Examples

| Material ID | Description | Source |
|---|---|---|
| DGEBPA | Diglycidyl ether of bisphenol A resin, | HEXION INC. |
| | commercially available as Epon resin | Columbus, Ohio, USA |
| | 828. | |
| BGE | Butyl glycidyl ether resin, | HEXION INC. |
| | commercially available as HELOXY | Columbus, Ohio, USA |
| | Modifier 61. | |
| CDMDGE | Cyclohexane dimethanol diglycidyl | HEXION INC. |
| | ether, | Columbus, Ohio, USA |
| | commercially available as HELOXY | |
| | 107. | |
| DGEBPA-PO | Propoxylated Diglycidyl ether of | ADEKA |
| | bisphenol A resin, commercially | CORPORATION, |
| | available as EP-4000S. | Tokyo, Japan. |
| E-1001F | Epoxy resin derived from a liquid | HEXION INC. |
| | epoxy resin and bisphenol-A, | Columbus, Ohio, USA |
| | commercially available as EPON Resin | |
| | 1001F. | |
| MY 0510 | N,N-Diglycidyl-4-glycidyloxyaniline, | HUNTSMAN |
| | commercially available as MY 0510. | ADVANCED |
| | | MATERIALS |
| | | AMERICAS LLC, The |
| | | Woodlands, TX, USA |
| 1,6HDT | 1,6-Hexanedithiol. | SIGMA-ALDRICH |
| | | INC, St. Louis, MO, |
| | | USA |
| GLYMO | Glycidyl 3-(trimethoxysilyl)propyl | EVONIK |
| | ether, commercially available as | CORPORATION, |
| | DYNASYLAN GLYMO. | Parsippany, NJ, USA |
| Dimer diamine | Diamine hardener, commercially | CRODA INC. |
| | available as PRIAMINE 1075. | Plainsboro, NJ, USA |
| DETA | Diethylene triamine hardener, | BASF. Florham Park, |
| | commercially available as DETA. | NJ, USA |
| TMPMP | Trimethylolpropane tris(3- | BRUNO BOCK, |
| | mercaptopropionate) hardener, | Teaneck, NJ, USA |
| | commercially available as THIOCURE | |
| | 430. | |
| PN-23J | Amine catalyst, commercially available | A&C CATALYSTS |
| | as AJICURE PN-23J | INC. Linden, NJ, USA |
| A-2442 | Epoxy polyamine adduct, commercially | EVONIK |
| | available as ANCAMINE 2442. | CORPORATION, |
| | | Parsippany, NJ, USA |
| F-1081 | Modified aliphatic polyamine, | T&K TOKA CO., |
| | commercially available as FUJICURE | LTD, Saitama, Japan |
| | FXR-1081. | |

### Example 1

The properties of a curable liquid feedthrough potting adhesive composition of the present disclosure were tested. A curable liquid feedthrough potting adhesive sample (Sample EX1) was prepared according to the compositions and methods of the present disclosure using an epoxy resin, a thiol hardener, and a solid catalyst, as described herein. A comparative sample (Sample CE) was also prepared using epoxy resins and amine hardeners.

Concentration (wt-%)

| Material ID | CE | EX1 |
|---|---|---|
| DGEBPA | 55.5 | 55 |
| BGE | 6.9 | — |
| Dimer diamine | 35.7 | — |
| DETA | 1.9 | — |
| TMPMP | — | 41 |
| PN-23J | — | 4 |

### Sample Preparation

Samples CE and EX1 were prepared using the materials and amounts shown. The materials were weighed, mixed, and then degassed under 50 mbar vacuum. The resulting samples was tested for pot life, thixotropic index, degree of cure after 2 hours, and glass transition temperature.

### Pot Life

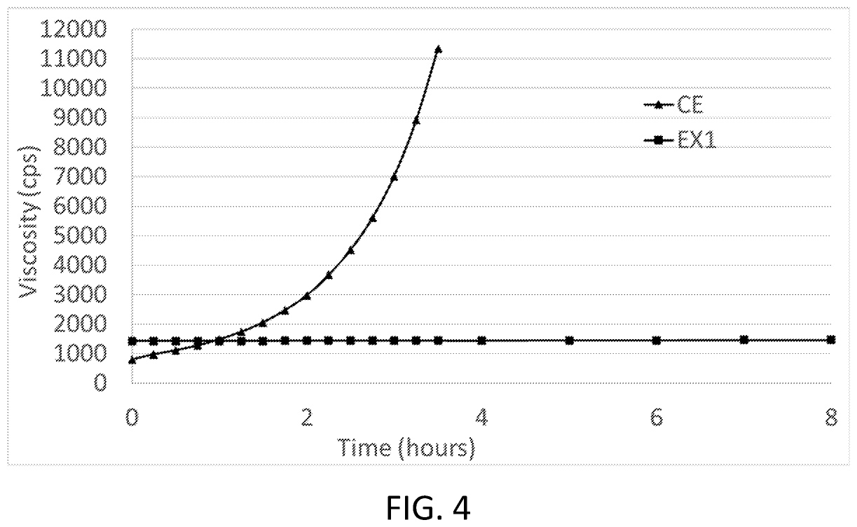

The pot life of each of samples CE and EX1 was determined as the time to reach a viscosity of 2,500 cps or higher at an ambient temperature of 23° C. Viscosity was measured at a shear rate of 10/s, according to ASTM D1084. The results are shown in FIG. 4. The pot life of EX1 was greater than 8 hours at an ambient temperature of 23° C. The viscosity of EX1 after 8 hours at 23° C. was 1,458 cps, which was only a 2% increase from the initially measured viscosity of 1,425 cps. The pot life of CE was less than 2 hours at an ambient temperature of 23° C. The viscosity of CE after 8 hours at 23° C. was 2,966 cps, which was a 276% increase from the initially measured viscosity of 789 cps.

### Thixotropic Index

Thixotropic index is defined as the ratio of the viscosity at 1/s shear rate over the viscosity at 10/s shear rate. A low thixotropic index of 1.0-1.2 may be desirable for feedthrough potting applications. A low thixotropic index of 1.0-1.2 may be described as indicating the liquid adhesive has free flowing and self-leveling properties. The measured thixotropic index of EX1 was 1.0. The measured thixotropic index of CE was 1.0.

Degree of Cure after 2 Hours at 50° C.

Degree of cure after 2 hours at an ambient temperature of 50° C. was calculated by the ratio of the measured enthalpy of each unreacted sample after 2 hours at an ambient temperature of 50° C., to the measured enthalpy of each unreacted sample after ramping ambient temperature at a rate of 10° C. per minute from 25° C. to 250° C. The enthalpy obtained by curing at the heating rate of 10° C. per minute from 25° C. to 250° C. may be described as fully cured (i.e., 100% cured) enthalpy. Enthalpy was measured by Differential Scanning calorimetry (DSC).

| | Enthalpy | Enthalpy after | |
|---|---|---|---|
| | after 2 | 10° C./min ramp | |
| | hours | from 25° C. | Degree |
| Sample | at 50° C. | to 250° C. | of cure |
| CE | 77 J/g | 313 J/g | 25% |
| EX1 | 406 J/g | 406 J/g | 100% |

### Glass Transition Temperature

The glass transition temperature (Tg) was determined by DSC, according to ASTM E1356. The glass transition temperature of fully-cured EX1 was 36° C. The glass transition temperature of fully-cured CE was 48° C.

### Example 2

The properties of curable liquid potting feedthrough adhesive compositions of the present disclosure were tested. Seven different curable liquid feedthrough potting adhesive samples (Samples EX1, EX2, EX3, EX4, EX5, EX6, and EX7) were prepared according to the compositions and methods of the present disclosure using epoxy resins, thiol hardeners, and solid catalysts, as described herein. A comparative sample (Sample CE) was also prepared using epoxy resins and amine hardeners.

| | Concentration (wt-%) | | | | | | | |
|---|---|---|---|---|---|---|---|---|
| Material ID | CE | EX1 | EX2 | EX3 | EX4 | EX5 | EX6 | EX7 |
| DGEBPA | 55.5 | 55 | 40 | 42 | 42 | 42.5 | 56.5 | 56.5 |
| BGE | 6.9 | — | — | — | — | — | — | — |
| CDMDGE | — | — | 7 | 6 | — | — | — | — |
| DGEBPA-PO | — | — | 7 | 7 | — | — | — | — |
| MY 0510 | — | — | — | — | 10 | 10 | — | — |
| E-1001F | — | — | — | — | — | — | 12.5 | 12.5 |
| 1,6HDT | — | — | — | — | — | — | 19 | 19 |
| GLYMO | — | — | 1 | 1 | 1 | 1 | 1 | 1 |
| Dimer diamine | 35.7 | — | — | — | — | — | — | — |
| DETA | 1.9 | — | — | — | — | — | — | — |
| TMPMP | — | 41 | 41 | 42 | 45 | 45.5 | 10 | 10 |
| PN-23J | — | 4 | — | — | — | — | — | — |
| A-2442 | — | — | 4 | 2 | 2 | 1 | 1 | — |
| F-1081 | — | — | — | — | — | — | — | 1 |

### Pot Life and Thixotropic Index

The pot life of each of Samples CE and EX1-EX7 was determined as the time to reach a viscosity of 2,500 cps or higher at an ambient temperature of 23° C. Pot life viscosity was measured at a shear rate of 10/s, according to ASTM D1084.

| | Pot Life at 23° C. | Thixotropic |
|---|---|---|
| Sample | (hours) | Index |
| CE | 2 | 1.0 |
| EX1 | 74 | 1.0 |
| EX2 | 240 | 1.0 |
| EX3 | 720 | 1.0 |
| EX4 | 480 | 1.0 |
| EX5 | 720 | 1.0 |
| EX6 | 240 | 1.0 |
| EX7 | 240 | 1.0 |

Degree of Cure after 2 Hours at 60° C.

Whether each sample was capable of fully curing within 2 hours at an ambient temperature of 60° C. was determined by comparing the measured enthalpy of unreacted material after 2 hours at an ambient temperature of 60° C. against the enthalpy of unreacted material after ramping ambient temperature at a rate of 10° C. per minute from 25° C. to 250° C. The enthalpy obtained by curing at the heating rate of 10° C. per minute from 25° C. to 250° C. may be described as fully cured (i.e., 100% cured) enthalpy. Enthalpy was measured by DSC.

| | CE | EX1 | EX2 | EX3 | EX4 | EX5 | EX6 | EX7 |
|---|---|---|---|---|---|---|---|---|
| Cured within 2 | No | Yes | Yes | No | Yes | No | Yes | Yes |
| hours at 60° C | | | | | | | | |

### Water Uptake

Water uptake of each sample was measured after each sample had soaked in 37° C. deionized (DI) water for 63 days. The water uptake was measured in accordance with ASTM D570-98.

| | CE | EX1 | EX2 | EX3 | EX4 | EX5 | EX6 | EX7 |
|---|---|---|---|---|---|---|---|---|
| Water uptake | 1.5 | 4.5 | 3.8 | 3.0 | 3.3 | 3.0 | 2.0 | 2.0 |
| (wt-%) | | | | | | | | |

It should also be noted that, as used in this specification and the appended claims, the phrase “configured” describes a system, apparatus, or other structure that is constructed to perform a particular task or adopt a particular configuration. The word “configured” can be used interchangeably with similar words such as “arranged”, “constructed”, “manufactured”, and the like.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

The following examples are a non-limiting list of clauses in accordance with one or more techniques of this disclosure.

Example 1. An implantable medical device feedthrough assembly comprising: a feedthrough ferrule; and a reaction product of a curable liquid feedthrough potting adhesive in the feedthrough ferrule, the adhesive comprising: an epoxy resin comprising one or more of a glycidyl ether group, an alkoxysilane group, and a nano silica; a thiol hardener comprising at least two thiol groups, an alkoxysilane group, or at least two thiol groups and an alkoxysilane group; and a solid catalyst comprising an amine group.

Example 2. The implantable medical device feedthrough assembly of Example 1, wherein the reaction product has a glass transition temperature of less than 50° C.

Example 3. The implantable medical device feedthrough assembly of Example 1, wherein the reaction product has a glass transition temperature of less than 40° C.

Example 4. The implantable medical device feedthrough assembly of claim 1, wherein the reaction product has a glass transition temperature of between 27° C. and 47° C.

Example 5. The implantable medical device feedthrough assembly of Example 1, wherein the reaction product is substantially free of water.

Example 6. The implantable medical device feedthrough assembly of Example 1, wherein the reaction product of the curable liquid feedthrough potting adhesive has a water uptake of less than 5 wt-% after 63 days in a 37° C. aqueous environment.

Example 7. The implantable medical device feedthrough assembly of Example 1, wherein the reaction product of the curable liquid feedthrough potting adhesive has a water uptake of less than 4 wt-% after 63 days in a 37° C. aqueous environment.

Example 8. The implantable medical device feedthrough assembly of Example 1, wherein the reaction product of the curable liquid feedthrough potting adhesive has a water uptake of less than 3 wt-% after 63 days in a 37° C. aqueous environment.

Example 9. The implantable medical device feedthrough assembly of Example 1, wherein the reaction product of the curable liquid feedthrough potting adhesive has a water uptake of less than 2 wt-% after 63 days in a 37° C. aqueous environment.

Example 10. The implantable medical device feedthrough assembly of Example 1, wherein the reaction product is curable within 2 hours at an ambient temperature of less than 70° C.

Example 11. The implantable medical device feedthrough assembly of Example 1, wherein the reaction product is curable within 3 hours at an ambient temperature of less than 60° C.

Example 12. The implantable medical device feedthrough assembly of Example 1, wherein the reaction product is curable within 4 hours at an ambient temperature of less than 50° C.

Example 13. The implantable medical device feedthrough assembly of Example 1, wherein the epoxy resin is non-polar.

Example 14. The implantable medical device feedthrough assembly of Example 1, wherein the epoxy resin has an octanol-water partition coefficient (log P) of greater than 2.

Example 15. The implantable medical device feedthrough assembly of Example 1, wherein the thiol hardener is non-polar.

Example 16. The implantable medical device feedthrough assembly of Example 1, wherein the thiol hardener has an octanol-water partition coefficient (log P) of greater than 2.

Example 17. The implantable medical device feedthrough assembly of Example 1, wherein the curable liquid feedthrough potting adhesive comprises less than 5% of the solid catalyst.

Example 18. The implantable medical device feedthrough assembly of Example 1, wherein the curable liquid feedthrough potting adhesive comprises less than 3% of the solid catalyst.

Example 19. The implantable medical device feedthrough assembly of Example 1, wherein the curable liquid feedthrough potting adhesive comprises less than 2% of the solid catalyst.

Example 20. A method for forming an implantable medical device, the method comprising: bonding a feedthrough pin within a ferrule with a curable liquid feedthrough potting adhesive, the adhesive comprising: (i) an epoxy resin comprising one or more of a glycidyl ether group, an alkoxysilane group, and a nano silica; (ii) a thiol hardener comprising at least two thiol groups, an alkoxysilane group, or at least two thiol groups and an alkoxysilane group; and (iii) a solid catalyst comprising an amine group; and curing the curable liquid feedthrough potting adhesive.

Example 21. The method of Example 20, wherein the curing the curable liquid potting adhesive occurs within 2 hours at an ambient temperature of less than 70° C.

Example 22. The method of Example 20, wherein the curing the curable liquid potting adhesive occurs within 3 hours at an ambient temperature of less than 60° C.

Example 23. The method of Example 20, wherein the curing the curable liquid potting adhesive occurs within 4 hours at an ambient temperature of less than 50° C.

Example 24. The method of Example 20, wherein the curing the curable liquid potting adhesive forms a cured potting adhesive with a glass transition temperature of less than 50° C.

Example 25. The method of Example 20, wherein the curing the curable liquid potting adhesive forms a cured potting adhesive with a glass transition temperature of less than 40° C.

Example 26. The method of Example 20, wherein the curing the curable liquid potting adhesive forms a cured potting adhesive with a glass transition temperature of between 27° C. and 47° C.

Example 27. The method of Example 20, wherein the curing the curable liquid potting adhesive forms a cured potting adhesive substantially free of water.

Example 28. The method of Example 20, wherein the curing the curable liquid potting adhesive forms a cured potting adhesive with a water uptake of less than 5 wt-% after 63 days in a 37° C. aqueous environment.

Example 29. The method of Example 20, wherein the curing the curable liquid potting adhesive forms a cured potting adhesive with a water uptake of less than 4 wt-% after 63 days in a 37° C. aqueous environment.

Example 30. The method of Example 20, wherein the curing the curable liquid potting adhesive forms a cured potting adhesive with a water uptake of less than 3 wt-% after 63 days in a 37° C. aqueous environment.

Example 31. The method of Example 20, wherein the curing the curable liquid potting adhesive forms a cured potting adhesive with a water uptake of less than 2 wt-% after 63 days in a 37° C. aqueous environment.

Example 32. The method of Example 20, wherein the epoxy resin is non-polar.

Example 33. The method of Example 20, wherein the epoxy resin has an octanol-water partition coefficient (log P) of greater than 2.

Example 34. The method of Example 20, wherein the thiol hardener is non-polar.

Example 35. The method of Example 20, wherein the thiol hardener has an octanol-water partition coefficient (log P) of greater than 2.

Example 36. The method of Example 20, wherein the curable liquid feedthrough potting adhesive comprises less than 5% of the solid catalyst.

Example 37. The method of Example 20, wherein the curable liquid feedthrough potting adhesive comprises less than 3% of the solid catalyst.

Example 38. The method of Example 20, wherein the curable liquid feedthrough potting adhesive comprises less than 2% of the solid catalyst.

Example 39. A curable liquid feedthrough potting adhesive suitable for use in a feedthrough ferrule of an implantable medical device, capable of curing within 3 hours at an ambient temperature of 70° C. or less, and with a pot life of at least 6 hours, the curable liquid feedthrough potting adhesive comprising: an epoxy resin comprising one or more of a glycidyl ether group, an alkoxysilane group, and a nano silica; a thiol hardener comprising two or more thiol groups, an alkoxysilane group, or two or more thiol groups and an alkoxysilane group; and a solid catalyst comprising an amine group.

Example 40. The curable liquid feedthrough potting adhesive of Example 42, wherein the adhesive is substantially free of water.

Example 41. The curable liquid feedthrough potting adhesive of Example 42, wherein a reaction product of the epoxy resin, the thiol hardener, and the solid catalyst, when cured, has a water uptake of less than 5 wt-% after 63 days in a 37° C. aqueous environment.

Example 42. The curable liquid feedthrough potting adhesive of Example 42, wherein a reaction product of the epoxy resin, the thiol hardener, and the solid catalyst, when cured, has a water uptake of less than 4 wt-% after 63 days in a 37° C. aqueous environment.

Example 43. The curable liquid feedthrough potting adhesive of Example 42, wherein a reaction product of the epoxy resin, the thiol hardener, and the solid catalyst, when cured, has a water uptake of less than 3 wt-% after 63 days in a 37° C. aqueous environment.

Example 44. The curable liquid feedthrough potting adhesive of Example 42, wherein a reaction product of the epoxy resin, the thiol hardener, and the solid catalyst, when cured, has a water uptake of less than 2 wt-% after 63 days in a 37° C. aqueous environment.

Example 45. The curable liquid feedthrough potting adhesive of Example 42, wherein the epoxy resin is non-polar.

Example 46. The curable liquid feedthrough potting adhesive of Example 42, wherein the epoxy resin has an octanol-water partition coefficient (log P) of greater than 2.

Example 47. The curable liquid feedthrough potting adhesive of Example 42, wherein the thiol hardener is non-polar.

Example 48. The curable liquid feedthrough potting adhesive of Example 42, wherein the thiol hardener has an octanol-water partition coefficient (log P) of greater than 2.

Example 49. The curable liquid feedthrough potting adhesive of Example 42, wherein the adhesive comprises less than 15% polar compounds.

Example 50. The curable liquid feedthrough potting adhesive of Example 42, wherein the adhesive has a pH of greater than 7.

Example 51. The curable liquid feedthrough potting adhesive of Example 42, comprising less than 5% of the solid catalyst.

Example 52. The curable liquid feedthrough potting adhesive of Example 42, comprising less than 3% of the solid catalyst.

Example 53. The curable liquid feedthrough potting adhesive of Example 42, comprising less than 2% of the solid catalyst.

Example 54. The curable liquid feedthrough potting adhesive of Example 42, wherein the adhesive is capable of curing within 2 hours at an ambient temperature of less than 60° C.

Example 55. The curable liquid feedthrough potting adhesive of Example 42, wherein the adhesive has a viscosity of less than 2,000 cps at an ambient temperature of between 20° C. and 25° C.

Example 56. The curable liquid feedthrough potting adhesive of Example 42, wherein the adhesive has a viscosity of less than 2,500 cps after 8 hours at an ambient temperature of between 20° C. and 25° C.

1. A curable liquid feedthrough potting adhesive suitable for use in a feedthrough ferrule of an implantable medical device, capable of curing within 3 hours at an ambient temperature of 70° C. or less, and with a pot life of at least 6 hours, the curable liquid feedthrough potting adhesive comprising:
an epoxy resin comprising one or more of a glycidyl ether group, an alkoxysilane group, and a nano silica;
a thiol hardener comprising two or more thiol groups, an alkoxysilane group, or two or more thiol groups and an alkoxysilane group; and
a solid catalyst comprising an amine group.

2. The curable liquid feedthrough potting adhesive of claim 1, wherein the adhesive is substantially free of water.

3. The curable liquid feedthrough potting adhesive of claim 1, wherein the curable liquid feedthrough potting adhesive is configured to form a cured reaction product having a water uptake of less than 5 wt-% after 63 days in a 37° C. aqueous environment.

4. The curable liquid feedthrough potting adhesive of claim 2, wherein the reaction product has a water uptake of less than 2 wt-% after 63 days in a 37° C. aqueous environment.

5. The curable liquid feedthrough potting adhesive of claim 1, wherein the epoxy resin is non-polar.

6. The curable liquid feedthrough potting adhesive of claim 1, wherein the epoxy resin has an octanol-water partition coefficient (log P) of greater than 2.

7. The curable liquid feedthrough potting adhesive of claim 1, wherein the thiol hardener is non-polar.

8. The curable liquid feedthrough potting adhesive of claim 1, wherein the thiol hardener has an octanol-water partition coefficient (log P) of greater than 2.

9. The curable liquid feedthrough potting adhesive of claim 1, wherein the adhesive has a pH of greater than 7.

10. The curable liquid feedthrough potting adhesive of claim 1, wherein the adhesive is capable of curing within 2 hours at an ambient temperature of less than 60° C.

11. An implantable medical device feedthrough assembly comprising:
a feedthrough ferrule; and
a curable feedthrough potting adhesive disposed on the feedthrough ferrule, wherein the curable feedthrough potting adhesive is capable of curing within 3 hours at an ambient temperature of 70° C. or less, and with a pot life of at least 6 hours, the curable liquid feedthrough potting adhesive comprising:
an epoxy resin comprising one or more of a glycidyl ether group, an alkoxysilane group, and a nano silica;
a thiol hardener comprising two or more thiol groups, an alkoxysilane group, or two or more thiol groups and an alkoxysilane group; and
a solid catalyst comprising an amine group.

12. The implantable medical device feedthrough assembly of claim 11, wherein at least one of the epoxy resin or the thiol hardener is non-polar.

13. The implantable medical device feedthrough assembly of claim 11, wherein at least one of the epoxy resin or the thiol hardener has an octanol-water partition coefficient (log P) of greater than 2.

14. The implantable medical device feedthrough assembly of claim 11, wherein the curable liquid feedthrough potting adhesive is configured to form a cured reaction product having a water uptake of less than 5 wt-% after 63 days in a 37° C. aqueous environment.

15. A method for forming an implantable medical device, the method comprising:
providing a ferrule defining a via and a feedthrough pin positioned within the via of the ferrule,
depositing a curable liquid feedthrough potting adhesive adjacent to and in contact with both the feedthrough pin and the ferrule, the curable liquid feedthrough potting adhesive comprising:
(i) an epoxy resin comprising one or more of a glycidyl ether group, an alkoxysilane group, and a nano silica;
(ii) a thiol hardener comprising at least two thiol groups, an alkoxysilane group, or at least two thiol groups and an alkoxysilane group; and
(iii) a solid catalyst comprising an amine group,
wherein the curable feedthrough potting adhesive is capable of curing within 3 hours at an ambient temperature of 70° C. or less, and with a pot life of at least 6 hours; and
curing the curable liquid feedthrough potting adhesive to form a reaction product bonded to both the feedthrough pin and the ferrule.

16. The method of claim 15, wherein at least one of the epoxy resin or the thiol hardener is non-polar.

17. The method of claim 15, wherein at least one of the epoxy resin or the thiol hardener has an octanol-water partition coefficient (log P) of greater than 2.

18. The method of claim 15, wherein the adhesive has a viscosity of less than 2,000 cps at an ambient temperature of between 20° C. and 25° C.

19. The method of claim 15, wherein the reaction product has a glass transition temperature of less than 50° C.

20. The method of claim 15, wherein the reaction product has water uptake of less than 5 wt-% after 63 days in a 37° C. aqueous environment.
